(19) United States
(12) Patent Application Publication
(10) Pub. No.: US 2007/0083081 A1
(43) Pub. Date: Apr. 12, 2007

(54) ENDOSCOPE

(75) Inventors: Frank SCHLAGENHAUF, Neulingen (DE); Jorg DIENER, Oberderdingen (DE)

Correspondence Address: AKIN GUMP STRAUSS HAUER & FELD L.L.P., ONE COMMERCE SQUARE, 2005 MARKET STREET, SUITE 2200, PHILADELPHIA, PA, 19103, US

(73) Assignee: RICHARD WOLF GMBH, Knittlingen (DE)

(21) Appl. No.: 11/539,992

(22) Filed: Oct. 10, 2006

(30) Foreign Application Priority Data
Oct. 11, 2005 (DE) 10 2005 049 021.2

(51) Int. Cl. A61B 1/00

(52) U.S. Cl. 600/104

(57) ABSTRACT

An endoscope is provided with a processing tool (24) arranged at its distal end. The processing tool can be moved by a mobile hollow shaft (12) extending in the longitudinal direction (X) of the endoscope. An endoscope optic (14) is arranged inside the mobile hollow shaft (12).

### BACKGROUND OF THE INVENTION

The invention relates to an endoscope or technoscope.

In particular, in the industrial environment it happens frequently that construction parts, for example cast parts, need to be tested and, if necessary, post-processed, e.g., lateral bores in pump housings or ball channels in motor blocks. During and after the post-processing, additional tests are necessary in order to determine if post-processing has been successful. This necessitates a frequent exchange of the tools and instruments used during the tests and post-processing.

### BRIEF SUMMARY OF THE INVENTION

An object of the invention is to provide an endoscope or technoscope allowing a simplified testing and processing of work pieces.

This object is attained by an endoscope having a processing tool arranged at its distal end, which is mobile via a mobile hollow shaft extending in the longitudinal direction of the endoscope, and having an endoscope optic arranged inside the mobile hollow shaft. Preferred embodiments are described below and in the dependent claims.

Although in the following the endoscope according to the invention is primarily described for use in technical applications, the endoscope according to the invention may also be used in the fields of human and veterinary medicine, as well as dentistry.

The endoscope according to the invention is provided with a processing tool at its distal end. With this processing tool the desired processes and/or post-processes can be performed on a work piece. For this purpose, the processing tool is constructed to be mobile and/or movable. A mobile hollow shaft is provided, extending in the longitudinal direction of the endoscope, i.e., from the proximal to the distal end thereof, for driving and/or moving the processing tool. The processing tool is set in motion and/or driven via the movement of the hollow shaft.

Further, according to the invention, a preferably fixed endoscope optic is arranged in the interior of the mobile hollow shaft. Using this endoscope optic, the area at the distal end of the endoscope and/or the area around the distal end of the endoscope can be observed, as known from conventional endoscopes. The endoscope optic may be fixed, i.e., rigidly connected to the housing of the endoscope. Alternatively, the endoscope optic may also be constructed to be rotatable, in order to move the view field of the endoscope optic in the hollow space to be examined by rotating the endoscope optic. However, the endoscope optic according to the invention is fixed in such a manner that it is not moved together with the hollow shaft when the schaft is driving the processing tool.

By combining a mobile processing tool and an endoscope optic according to the invention, it is possible to simultaneously perform an optical supervision and a processing of the work piece. Furthermore, due to the concentric arrangement of the hollow shaft and the endoscope optic, a very compact design of the endoscope is possible with a very small diameter, so that even very small hollow spaces can be inspected and in particular processed under visual supervision.

Preferably, the hollow shaft is mobile in the longitudinal direction of the endoscope and/or rotational around its longitudinal axis. An oscillating motion of the processing tool in the longitudinal direction of the endoscope can be achieved by the motion in the longitudinal direction. A rotating motion of the processing tool can be achieved by rotation around the longitudinal axis of the hollow shaft. The rotation of the hollow shaft, and thus the rotation of the processing tool as well, occurs preferably such that a rotation of more than 360° is performed in one rotational direction, preferably a continuous rotation in the same rotational direction around the longitudinal axis. In special embodiments it is also possible to overlap both movements, so that the processing tool simultaneously oscillates and rotates. Further, it is not mandatory for the processing tool to perform the same type of motion as the hollow shaft, rather a transfer of an oscillating motion of the hollow shaft, for example, into a rotational motion can also be achieved here, for example an oscillating rotating motion of the processing tool.

Preferably, the hollow shaft has a circular cross-section. Correspondingly, it is further preferred that in the interior of the hollow shaft the endoscope optic have a corresponding cross-section, so that a compact concentric arrangement of the hollow shaft and the endoscope optic is possible. The circular cross-section of the hollow shaft is particularly suitable for rotational movements of the hollow shaft around its longitudinal axis.

It is expedient that bearing elements be arranged between an inner wall of the hollow shaft and an outer wall of the endoscope optic. These bearing elements ensure precise guidance and/or support of the rotating hollow shaft at the fixed endoscope optic. For this purpose, preferably at least at the proximal and at the distal end, bearing elements are provided between the endoscope optic and the hollow shaft. A constant, defined alignment of the endoscope optic relative to the hollow shaft is ensured by the bearing elements, so that a precise observation of the processing by the processing tool is always ensured via the endoscope optic.

Furthermore, a drive for moving the hollow shaft is preferably provided at the proximal end of the endoscope. This may be an electric motor, for example. Alternatively, a coupling may be provided at the proximal end, to which a standardized drive, for example an electric motor and/or hollow shaft motor, or a shaft can be coupled for a connection to an external drive.

According to a particular embodiment of the invention, the hollow shaft may be formed to be flexible. For example, the hollow shaft may be formed as a flexible shaft, such that even the processing of hard to reach spaces or places is possible using the endoscope. Here, the flexible embodiment of the hollow shaft is made such that a movement for driving the processing tool, in particularly a rotation, remains possible.

Correspondingly, the endoscope optic may be formed to be flexible as well, in order to be arranged in a flexible hollow shaft. For example, the endoscope optic may be bent and/or experience curving together with the flexible hollow shaft in order to process and optically examine hard to reach places.

According to another preferred embodiment, at the distal end of the hollow shaft a coupling is formed for accepting the processing tool. Such a coupling allows various processing tools to be connected to the hollow shaft in an exchangeable manner. For example, the processing tool can be selected and connected to the hollow shaft depending on the process to be performed. Further, it is possible to exchange the processing tool when worn.

Preferably a transmission, in particular an angle drive, is arranged at the distal end of the hollow shaft, by which the processing tool can be moved. Such a transmission allows, on the one hand, a transfer and transmission of the movement of the hollow shaft to move the processing tool. Further, the direction of the movement can be changed, for example a linearly oscillating motion of the hollow shaft can be transferred into a rotational motion, particularly into an oscillating rotational motion of the processing tool. Alternatively, it is also possible to transfer a spinning and/or rotational motion of the hollow shaft via the transmission into an oscillating linear motion of the processing tool, for example via an eccentric shaft or a camshaft.

Further the transmission may be formed as an angle drive, so that it is possible for the rotational axis of the processing tool to extend at an angle relative to the rotational and/or longitudinal axis of the hollow shaft. For example, the rotational axis of the processing tool may be angled by 90° relative to the rotational axis of the hollow shaft. This may also be achieved by a spur gear or a miter gear. Here, the sprocket and/or miter arranged at the hollow shaft is preferably formed as hollow in its center, so that the endoscope optic can extend through the area, or at least the view field of the endoscope optic may extend through this area. This allows the observation of the distal end through the transmission.

In order to adjust the direction of the observation and/or the view field of the endoscope optic to the intended purpose, the viewing window of the endoscope optic may be arranged at the distal end in an angled manner, so that the direction of the view field is not in the distal direction, but angled relative to the longitudinal axis of the endoscope, for example by 45° in the radial direction. Additionally, the viewing window may be arranged such that the view field is directed in the radial direction, i.e., the optical axis of the view field extends normally relative to the longitudinal axis of the endoscope and/or the longitudinal axis of the hollow shaft. Correspondingly, at the distal end of the endoscope optic a prism or another suitable optical element may be arranged for an appropriate deflection of the radiation path.

According to another preferred embodiment, at least one probe channel is formed on the hollow shaft extending in the longitudinal direction of the endoscope. Here, it is particularly preferred for the probe channel to be formed such that it is connected to the hollow shaft in a fixed manner, i.e., that it moves together with the hollow shaft. This probe channel allows the guidance or holding of processing tools. For example, the probe channel may be open towards the distal end of the hollow shaft and, for example, a sanding wire, which rotates together with the hollow shaft, may be used in the probe channel. When the probe channel extends to the proximal end, it is further possible for the processing tool, for example a sanding wire, to be withdrawn from the proximal end when worn.

Alternatively, a rod- or wire shaped processing tool in the probe channel can also be moved together with the hollow shaft in the longitudinal direction of the endoscope in an oscillating manner, in order to allow a desired processing on a work piece at the distal end of the endoscope and/or in the area of the distal end of the endoscope. Here, it is also possible for the processing tool to be withdrawn from the proximal end when the probe channel extends to the proximal end. Further preferred, it is also possible to provide more than one probe channel on the hollow shaft. The probe channels are preferably formed in the area of the circumferential wall of the hollow shaft, i.e., they extend eccentrically relative to the hollow shaft.

According to another preferred embodiment, the processing tools is formed directly at the distal end of the hollow shaft. In particular, the circumferential wall of the hollow shaft may itself form the processing tool at the distal end. For example, this area of the hollow shaft may be specially cut or formed in another suitable way to form a milling or cutting tool. Here, the material of the hollow shaft may also be appropriately hardened or coated, for example by hard metal, ceramics, diamond, or the like.

The form of the distal end of the hollow shaft may also be adapted to the processing task, for example annular or conical. Here, almost all forms and coatings of processing tools can be used for cutting, in particular. For example, it is possible to circumferentially coat the hollow shaft at the distal end with diamond, in order to allow work pieces to be cut when the coated surface rotates or oscillates.

For example, the processing tool can be embodied as a sawing, milling, and/or cutting tool. In this manner, it is possible, in particular, to form the processing tool for all types of cutting, either with a geometrically determined blade or an unspecified one.

According to another preferred embodiment, the hollow shaft is formed to be removable and/or exchangeable. It allows, on the one hand, disassembly of the entire endoscope for cleaning and maintenance purposes. Preferably, the endoscope optic may also be formed removable and/or exchangeable. On the other hand, this embodiment allows the exchange of the hollow shaft, for example when damaged or in order to connect various hollow shafts to the very same endoscope. In this manner, various hollow shafts can be provided for different processing tasks, and can then be connected to the endoscope as desired. For example, it is also possible optionally to provide a flexible or a rigid endoscopic shaft and/or hollow shaft with the corresponding endoscope optic. Further, if the processing tool is formed directly at the distal end or connected to the distal end in a fixed manner, the hollow shaft may be formed as a single-use part, which is completely replaced by a new hollow shaft with a new processing tool, when the processing tool is worn.

The endoscope optic may generally be formed as any known endoscope optic. This may be, for example, a system of lenses, a fiber optic system, or even a camera system, which allows an image transmission from the distal end of the endoscope to the proximal end and/or display means at a farther distance. With a camera system it is particularly preferred for the camera to be arranged immediately in the area of the distal end and to provide an image transmission via electrical signals with the appropriate connection wires then extending through a shaft to the proximal end of the endoscope. The shaft is then constructed as a fixed optic shaft, which forms the endoscope optic, i.e., the optic shaft is not movable together with the hollow shaft during processing.

At the distal end of the endoscope it is further preferred for at least one recess to be formed in the wall of the hollow shaft, which recess is located in the view field of the endoscope optic or is movable into the view field of the endoscope optic. This means that the viewing window of the endoscope optic at the distal end is preferably so aligned that it faces the recess. This arrangement allows observations by the endoscope optic through the hollow shaft, namely through the recess.

Depending on the type of motion and the direction of the hollow shaft, it is not always possible to design a recess such that the recess is in the view field of the endoscope optic over the entire motion path of the hollow shaft so that a continuous observation is possible through the recess. This is particularly the case in rotational embodiments of the processing tool and the hollow shaft. In these embodiments one recess or several recesses may be provided, which are arranged such that they periodically scan the view field of the endoscope optic during the motion of the hollow shaft and/or the processing tool. In a rapid movement, in particular a rapid rotation of the processing tool or the hollow shaft, this allows a continuous observation via the endoscope optic, because the sluggishness of the eye does not recognize the interruptions of the view field by the wall of the hollow shaft and/or by the massive parts of the processing tool.

Depending on the arrangement and design of the processing tool at the distal end of the hollow shaft, the recess and/or recesses can be arranged in the wall of the hollow shaft and/or in the processing tool.

According to a special embodiment, at least one recess is formed in the circumferential wall of the hollow shaft, and the view field of the endoscope optic is at least partially aligned in the radial direction relative to the longitudinal axis of the endoscope. This means that the endoscope optic allows an observation in the radial direction and/or at an angle relative to the longitudinal axis of the endoscope through the circumferential wall of the hollow shaft, when the recess lies in the view field of the endoscope optic and/or passes through the view field of the endoscope optic during the movement of the hollow shaft.

In case the capturing of images via the endoscope optic occurs with a camera, it is preferred that this image capturing with a camera be synchronized with the movement of the hollow shaft. This means that in the case that one or more recesses are provided in the processing tool or the wall of the hollow shaft, which periodically pass through the view field of the camera and/or the endoscope optic, the shutter speed of the camera is synchronized or programmed such that the camera always captures a picture precisely at the moment the recess passes through the view field of the camera and/or the endoscope optic. If the movement of the hollow shaft and/or the processing tool and the correspondingly synchronized camera is sufficiently fast, images thus captured appear constant to the human eye.

According to another preferred embodiment, at least one rinse channel is formed between the outer wall of the endoscope optic and the inner wall of the hollow shaft. This rinse channel is preferably concentric relative to the hollow shaft and the endoscope optic and allows feeding of a fluid, for example a gas or a rinsing liquid, from the proximal end of the endoscope, in order to clear the processing area, in which the processing tool moves, from contaminants and in particular from chips created by the processing tool. In this manner, particularly the viewing window of the endoscope optic at the distal end can be kept free from contaminants by a rinsing agent, for example a rinsing liquid or a rinsing gas.

According to a particular embodiment, it is possible to arrange several rinse channels between the endoscope optic and the hollow shaft. For this purpose, another shaft can be arranged between the endoscope optic and the hollow shaft, such that between the shaft and the endoscope optic a free space remains, and between the shaft and the inner wall of the hollow shaft a second free space remains. In this manner, two channels are created positioned inside one another, preferably concentrically. For example, a rinsing liquid can be fed through one of these channels and can be removed through the other channel.

This additional sheath and/or intermediate wall is preferably formed in a locally fixed manner relative to the endoscope optic. However, it may also be formed locally fixed relative to the hollow shaft, so that it moves together with the hollow shaft. The latter embodiment particularly allows a rinsing liquid to be fed and removed always at a defined location of the processing tool, independent of the present position of the processing tool on its motion path, in particular of the present angular position of the processing tool. Instead of forming the rinse channels in a concentric manner, i.e., as annular channels, it is also possible to provide rinse channels eccentrically relative to the longitudinal axis of the endoscope on the endoscope optic or on the hollow shaft.

According to another preferred embodiment, a light sheath is provided for lighting the space surrounding the distal end of the endoscope. This particularly refers to the space in the view field of the endoscope optic. For this purpose, the light sheath is usefully provided with a reflective surface aligned in the distal direction in the direction of the view field.

The light sheath is here preferably arranged outside the hollow shaft. Alternatively, it is also possible, however, to arrange the light sheath inside the hollow shaft, for example on the endoscope optic. The light sheath may preferably be formed as a light guide. In this embodiment the light source itself, i.e., the lighting means, is preferably arranged at the proximal end of the endoscope or can be coupled thereto. This allows a very slim embodiment of the entire endoscope at its distal end, because the light sheath and/or its light guide can be formed very thin. Alternatively or additionally, it is also possible to arrange at least one light means directly in the light sheath. The use of light diodes as lighting means is particularly preferred, which diodes can be formed very small and furthermore create only very little heat.

The light sheath is preferably formed with an annular cross-section, so that it can be arranged concentrically relative to the endoscope optic and/or to the hollow shaft. This facilitates a very small diameter of the entire endoscopic shaft. Furthermore, an even illumination of the space at the distal side of the endoscope can be achieved.

The light sheath can be formed to be movable together with the hollow shaft, i.e., the light sheath rotates together with the hollow shaft. This requires sliding contacts for transferring energy, in case electric lighting means are arranged in the light sheath, which preferably are provided in the area of the proximal end of the endoscope. In case the light sheath is formed as a pure light guide, a light connection is provided, preferably at the proximal end of the endoscope, which allows a rotation or linear motion of the light sheath relative to a fixed light source and/or a fixed light guide. For example, the proximal end face of the light sheath may be located opposite to a parallel exit surface of a fixed light guide, so that light can be transmitted from the fixed light guide to the moving light sheath.

Alternatively, it is also possible to arrange the light sheath to be mobile relative to the hollow shaft. This allows formation of the light sheath in a fixed manner relative to the endoscope optic and the other components of the endoscope, so that the hollow shaft can be moved relative to both the light sheath and the endoscope optic.

According to another preferred embodiment, the hollow shaft is surrounded circumferentially by a protective sheath. This protective sheath can ensure that no body parts or work pieces, not intended to be worked upon, come into contact with the moving hollow shaft. In this manner, the protective sheath or a further protective sheath may also surround the processing tool in a partial region, for example in a circumferential segment, in order to prevent areas of the work piece not to be worked upon from coming into contact with the processing tool.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings:

FIG. 1 is a partially sectioned longitudinal side view of an endoscope according to a first embodiment of the invention;

FIG. 2 is a partially sectioned longitudinal side view of a flexible endoscope according to an embodiment of the invention;

FIG. 3 is a sectional side view of a shaft of an endoscope according to another embodiment of the invention;

FIG. 4 is a sectional side view of a shaft of an endoscope according to another embodiment of the invention;

FIG. 5 is a sectional side view of a shaft of an endoscope according to another embodiment of the invention;

FIG. 6 is a sectional side view of a shaft of an endoscope according to another embodiment of the invention;

FIG. 7 is a partially sectioned side view of a shaft of an endoscope according to another embodiment of the invention;

FIG. 8 is a side view of the distal end of a shaft of an endoscope according to another embodiment of the invention;

FIG. 9 is a partial sectional side view of a distal end of a shaft of an endoscope according to another embodiment of the invention;

FIG. 10 is a partial sectional side view of the distal end of a shaft of an endoscope according to another embodiment of the invention;

FIG. 11 is a partially sectioned side view of a shaft of an endoscope according to another embodiment of the invention;

FIG. 12 is a sectional side view of a shaft of an endoscope according to another embodiment of the invention;

FIG. 13 is a sectional side view of a shaft of an endoscope according to another embodiment of the invention; and

FIG. 14 is a sectional side view of the distal end of a shaft of an endoscope according to another embodiment of the invention.

In the following description various embodiments of the invention are described. Here, like parts are marked with the same reference numerals. Further, essentially only the differences between the individual embodiments are described. It should be understood here that details not explained in greater detail in the following embodiments may be formed according to the description of the other embodiments.

### DETAILED DESCRIPTION OF THE INVENTION

According to FIG. 1, first the general design of the endoscope according to the invention is described. The endoscope according to the invention comprises an endoscope housing 2 and an endoscope shaft 4 extending towards the distal end in the direction of the longitudinal axis X. The endoscope housing 2 is essentially embodied as those of known endoscopes and has a viewing opening 6 at the proximal end, optionally a light connection 8, as well as a connection for the rotational drive 10, not shown in FIG. 1. When a video optic is used, an electric connection for an external display may also be provided, in particular a monitor and/or video controller, instead of the viewing opening 6.

The form of the endoscope shaft 4 is essential for the invention, with the endoscope shaft 4 shown in FIG. 1 representing a fixed endoscope shaft 4, which extends in a straight fashion. The endoscope shaft 4 is preferably connected to the endoscope housing 2 in a detachable manner. This allows the connection of different endoscope shafts 4 to the very same endoscope housing 2, in order to allow the use of the endoscope for different purposes. It further allows the exchange of parts when worn and facilitates the cleaning and maintenance of the endoscope.

In the example shown in FIG. 1, the endoscope shaft 4 is formed by an outer hollow shaft 12 and by an endoscope optic 14 centrally arranged inside the hollow shaft 12. The hollow shaft 12 and the endoscope optic 14 extend parallel and concentrically relative to the longitudinal axis X. In the example shown, the endoscope optic 14 is formed as an optical lens as known from (conventional) endoscope optics. In this endoscope optic the optic elements are arranged in a rigid shaft. A free space 16 in the form of an annular space is formed between the hollow shaft 12 and the endoscope optic 14, which can serve as a rinse channel. In the free space 16, in the area of the distal end of the endoscope, a bearing element 18 is arranged, and a bearing element 20 is arranged in the area of the proximal end of the endoscope shaft 4. The bearing elements 18 and 20 are formed as slide bearings and allow a mobile guidance of the hollow shaft 12 at the outer circumference of the endoscope optic 14. Preferably, this guidance is essentially free from play.

When using the free space 16 as a rinse channel, fluid passages may be formed in the bearing elements 18 and 20 in the direction parallel to the longitudinal axis X. In the example shown the hollow shaft 12 is arranged rotating around the endoscope optic 14. The rotation occurs via the drive 10 at a gear 22, formed at the outer circumference of the hollow shaft 12, in the area of the proximal end of the hollow shaft 12. The distal end of the hollow shaft 12 forms a processing tool 24. In the example shown the distal end expands so that an annular bead forms. The bead may be coated at its outer circumferential surface, for example with diamonds, in order to form a cutting tool. Cutting can thus be performed with the processing tool 24 when the hollow shaft 12 is rotated.

The endoscope optic is arranged, in the example shown, such that the viewing window 26 is recessed in the proximal direction at the distal end of the endoscope optic 14 relative to the distal end of the hollow shaft 12. Thus, the viewing window 26 is arranged inside the hollow shaft 12 in a protected manner. In the example shown, the view field 28 is formed at an angle relative to the longitudinal axis X of the endoscope optic 14, i.e., the view field 28 is not arranged precisely in the distal direction but slightly pivoted in the radial direction. This can be achieved, for example, by a prism positioned in the distal end of the endoscope optic 14.

In the circumferential wall of the hollow shaft 12 recesses 30 are formed at a distance from the distal end of the hollow shaft 12 at the distal side of the viewing window 26. These recesses 30 allow, together with the openly formed distal end face 32 of the hollow shaft 12, an observation of the area surrounding the processing tool 24. This way, observations can be made through the endoscope optic 14, through the recesses 30, and the end face 32, in order to supervise the area of the work piece, which simultaneously can be processed by the processing tool 24. This allows processing under constant visual control. Bars 31 remain in the circumferential wall of the hollow shaft 12 between the circumferentially arranged recesses 30. Due to the rapid rotation of the hollow shaft 12 around the longitudinal axis X during processing, they do not interfere with the image, though, which the observer sees through the endoscope optic 14. Due to the sluggishness of the eye, the observer will not recognize these bars 31, which periodically pass the view field 28.

A gas or a fluid, for example, can be fed from the proximal end through the free space 16, in order to rinse the processing space at the distal end free from contaminants, in particular chips, which are generated by the processing tool 24, so that the viewing window 26 and the view field 28 are kept free from contaminants.

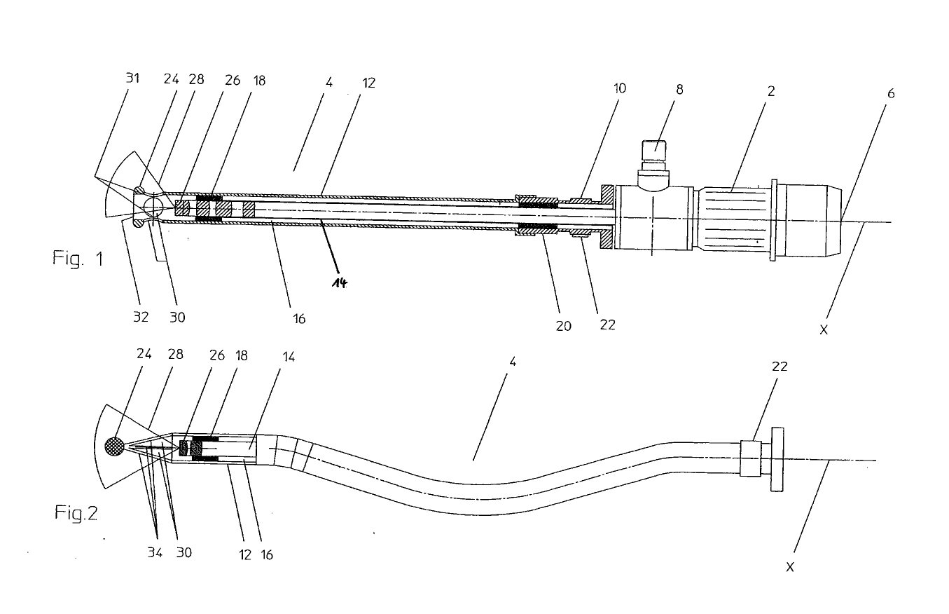

FIG. 2 shows a second embodiment of an endoscopic shaft 4. The endoscope housing 2 is not shown here, but it is constructed according to the above-mentioned description. This also applies for additional embodiments described in the following, in which only the endoscope shaft 4 is shown.

The endoscope shaft 4 according to FIG. 2 is formed to be flexible, i.e., the longitudinal axis X can be curved and/or bent as needed and does not necessarily extend in a straight manner. This allows the processing and observation of even hard to reach places, in particular it also allows the insertion of the endoscope shaft 4 into bent openings and channels. In order to implement the flexibility of the endoscope shaft 4, the hollow shaft 12 is formed as a flexible hollow shaft, for example as a flexible fiber optic.

Another difference of the embodiment according to FIG. 2 relative to the embodiment according to FIG. 1 is the form of the processing tool 24. The processing tool 24 in FIG. 2 is formed as a spherical milling and/or cutting head, which is connected via bars 34 to the distal end of the hollow shaft 12. The bars 34 extend conically and/or in a pyramid shape, such that they meet at the proximal side of the spherical head. Circumferential free spaces and/or recesses 30 are formed between the bars 34, which allow the view to the area around the processing tool 24 through the processing tool 24, as described in FIG. 1. In the example shown in FIG. 2 the view field 28 is aligned concentrically to the longitudinal axis X in the distal direction.

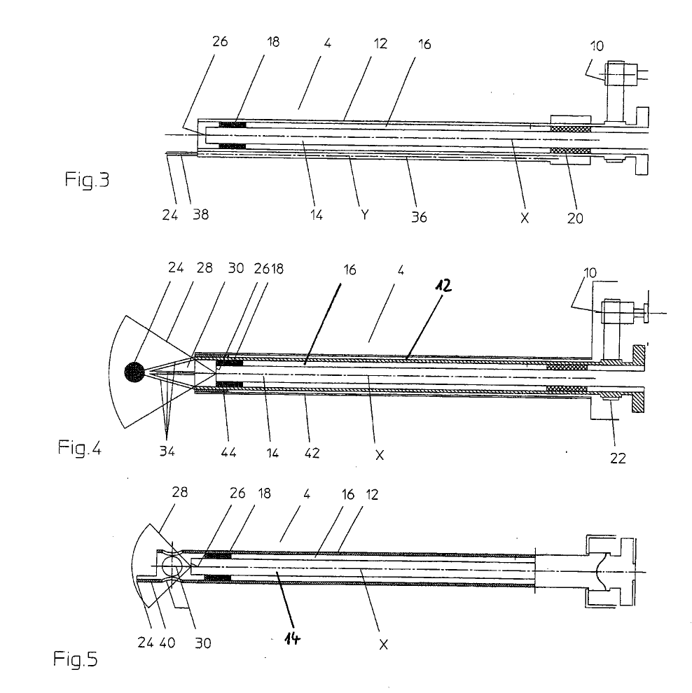

FIG. 3 shows another variant of the processing tool. According to FIG. 3, a probe channel 36 is formed on the hollow shaft 12, extending parallel to the longitudinal axis X on the circumferential wall of the hollow shaft 12. In the probe channel 36, with its longitudinal axis Y extending parallel to the longitudinal axis X of the endoscope shaft 4, a sanding wire 38 is used, which extends beyond the distal end of the hollow shaft 12 and the probe channel 36. This means that the sanding wire 38 protrudes in the distal direction at the distal end of the endoscope shaft 4. When the hollow shaft 12 rotates, the sanding wire 18 rotates on a circular path and thus forms the processing tool 24, which can be used for example for sanding and/or removing ridges. The endoscope optic 14 is provided with a viewing window 26 aligned in the distal direction, as described above, and allows a free view through the distal opening of the hollow shaft 12 to the area in which the processing is performed by the sanding wire 38. Here, also, the viewing window 26 is recessed in the proximal direction relative to the distal end of the hollow shaft 12.

FIGS. 3 and 4 further show the drive 10 as a belt drive, which drives the gear 22. Here, a toothed belt can be used. Alternatively, a different drive belt may also be used, with no gear 22, but rather a smooth driving wheel, for example for a V-belt, being used at the appropriate location. Any other type of appropriate rotational drives for the hollow shaft 12 can also be used here.

FIG. 5 shows an embodiment similar to the one in FIG. 3. Here, instead of a probe channel 36, a distally directed protrusion 40 is provided, which forms a pin and/or bar extending in the distal direction, which, e.g., is driven in an oscillating manner and thus forms a processing tool 24. For this purpose the protrusion 40 can be coated with diamonds, for example. In the embodiment according to FIG. 5, further recesses 30 are provided, as described according to FIG. 1.

FIG. 4 shows a processing tool corresponding to the processing tool 24 described according to FIG. 2. In contrast to FIG. 2, in the embodiment according to FIG. 4 the endoscope shaft 4 is formed to be rigid. Additionally, a protective sheath 42 is provided here, which circumferentially surrounds the hollow shaft 12 with a spacing. The protective sheath 42 is formed to be fixed, i.e., it does not rotate together with the hollow shaft 12. Appropriately, at the distal end and the proximal end a bearing element 44 is provided as a slide bearing between the rotating hollow shaft 12 and the fixed protective sheath 42. The protective sheath 42 prevents the rotating and/or moving hollow shaft 12 from coming into contact with the body parts or work piece parts and damaging them. Further, the danger of injuring an operator is minimized.

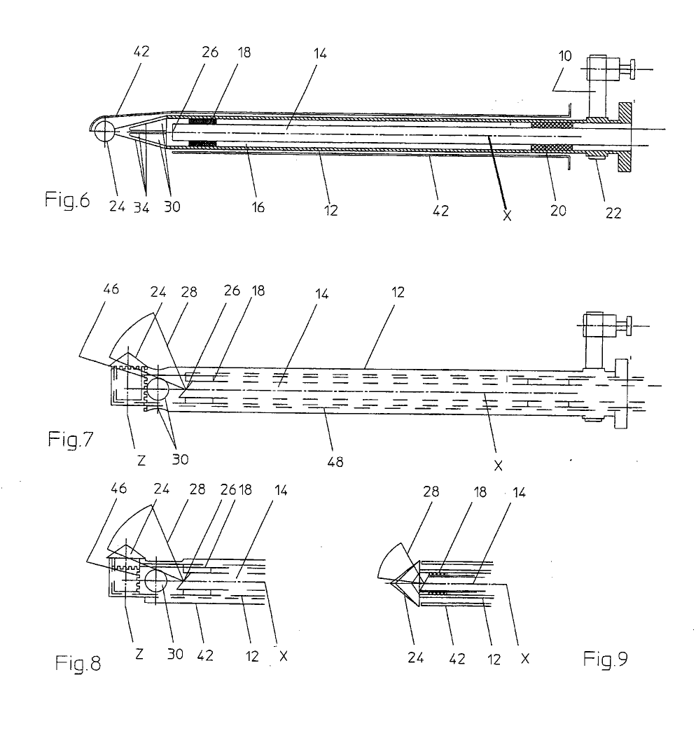

Another variant of the embodiment according to FIG. 4 is shown in FIG. 6. Here, the protective sheath 42 is further extended in the distal direction, so that it also surrounds the area of the bars 34 and the processing tool 24 at a peripheral side. Further, it extends at the distal end half way over the end face of the processing tool 24, so that the tool is free only in a limited circumferential area relative to the longitudinal axis and can perform processing here.

FIGS. 7 and 8 show embodiments in which the processing tool 24 is formed at the distal end conically and at an angle. The processing tool 24 according to FIGS. 7 and 8 is formed as a conical milling or cutting body, optionally with an appropriate surface coating. The rotational axis Z is angled by 90° relative to the longitudinal axis X, so that the processing tool 24 according to FIGS. 7 and 8 extends in the radial direction. The drive of the laterally aligned processing tool 24 occurs here by a pair of miter gears and/or spur-gears 46. Alternatively, this may also be embodied as a pair of friction gears. The processing tool 24 is held, according to the embodiment in FIG. 7, by a fixed sheath 48 arranged between the optic 14 and the hollow shaft 12. In the embodiment according to FIG. 8, the processing tool 24 is held by a protective sheath 42 circumferentially surrounding the hollow shaft 12 (similar to the embodiments in FIGS. 4 and 6).

In the embodiments according to FIGS. 7 and 8 the viewing window 26 and/or the view field 28 of the endoscope optic 14 is directed diagonally and/or laterally, such that the processing area can be observed radially in front of the processing tool 24.

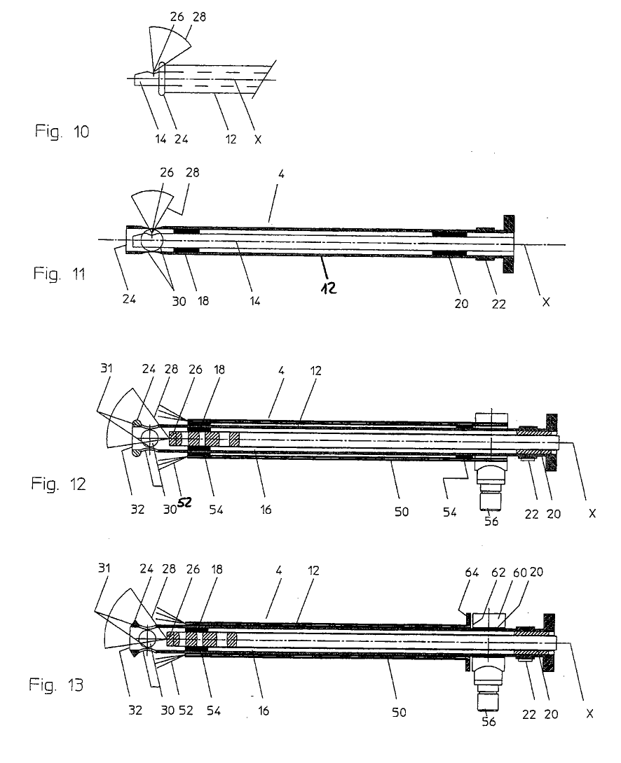

In the embodiment according to FIG. 9, the processing tool 24 is formed as a conical cutter and/or drill, with recesses being provided, distributed circumferentially, placed between the blades of the cutter. The recesses periodically pass by the view field 28 during rotation, so that the view through the cutter is possible, as described above. In the embodiment according to FIG. 9, once more a protective sheath 42 is provided surrounding the hollow shaft 12.

In the embodiment according to FIG. 10, at the distal side of the processing tool 24 the endoscope optic 14 extends beyond the distal end of the hollow shaft 12. The viewing window 26 is arranged such that the view field 28 points in the backward viewing direction, i.e., in the proximal direction angled relative to the longitudinal direction X. This means that the processing tool 24 is arranged at the proximal side of the viewing window 26. This allows observation of the processing tool 24 during its engagement in operation through the rearwardly directed view field 28.

The embodiment according to FIG. 11 corresponds essentially to the embodiment described according to FIG. 1, with the difference that the viewing window 26 of the endoscope optic 14 is arranged circumferentially, so that a radially directed view field 28 is achieved through the recesses 30 arranged circumferentially in the hollow shaft 12.

On the basis of FIGS. 12 and 13 two embodiments are described in which an additional illuminating device is provided. The arrangement according to FIG. 12 corresponds essentially to the arrangement described according to FIG. 1, with the view field in FIG. 12 being directed distally at a slight angle relative to the longitudinal axis X. The hollow shaft 12 is concentrically surrounded by a light sheath 50, which acts as a light guide in the direction from the proximal to the distal end of the endoscope shaft 4. Here, the distal end face of the light sheath 50 is formed as a light exit surface from which the light rays 52 exit in the distal direction in order to illuminate the view field 28 and the area around the processing tool 24. The light sheath 50 is designed to be fixed. For this purpose, bearing elements 54 are also provided between the inner circumference of the light sheath 50 and the outer circumference of the hollow shaft 12, so that the hollow shaft 12 can rotate relative to the light sheath 50. Preferably, the bearings 54 are also formed as slide bearings. At the proximal end of the light sheath 50 a light guide connection 56 is provided, through which light from an external light source can be introduced to the light sheath 50.

FIG. 13 shows another arrangement of the light sheath 50, in which the light sheath 50 is also arranged concentrically at the outer circumference of the hollow shaft 12, but directly and fixedly connected thereto, so that the light sheath 50 rotates together with the hollow shaft 12. The distal end face of the light sheath 50 is also formed as a light exit surface, so that the light rays 52 can exit in the distal direction in order to illuminate the view field 28 and the periphery of the processing tool 24. A coupling structure 58 is created between the rotating light sheath 50 and the light guide connector 56 in such a manner that a light-conducting sheath 60 is connected to the light conducting connector 56, with its distal end face 62 being formed as a light exit surface. Correspondingly, the proximal end face 64 of the light sheath 50, which here is additionally expanded in the radial direction relative to the light sheath 50, is formed as a light entry surface, so that the light from the fixed end faces 62 can be radiated into the rotating faces 64 and can then be conducted further via the light sheath 50 to the distal end of the endoscope shaft 4.

In the embodiment according to FIG. 13 another possible embodiment of the processing tool 24 is shown, which has an acute circumferential edge in contrast to the rounded embodiment according to FIGS. 1 and 12.

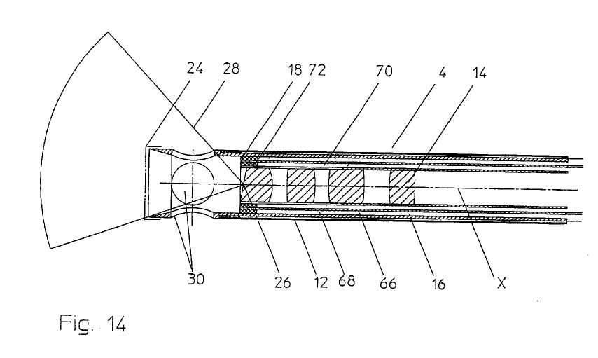

According to the embodiment in FIG. 14, the possible arrangement of two rinse channels is described between the endoscope optic 14 and the hollow shaft 12. The processing tool 24 formed by the edge of the distal end face of the hollow shaft 12 and the arrangement of the view field 28 and the viewing window 26 correspond to the previously described embodiments. According to the embodiment in FIG. 14, a sheath 66 is arranged in the free space 16 between the endoscope optic 14 and the inner wall of the hollow shaft 12, which divides the free space 16 into two annular channels 68 and 70. The channels 68 and 70 form two rinse channels with a rinsing liquid or rinsing gas being introduced from the proximal end through one of the channels, for example, and being removed though the other channel. Concentrically spaced by distancing elements 72 the sheath 66 is held on the endoscope optic 14, preferably in a non-rotating manner. The bearing elements 18 arranged between the sheath 66 and the inner wall of the hollow shaft 12 allow a rotation of the hollow shaft 12 relative to the sheath 66 and simultaneously hold the sheath 66 concentrically relative to the hollow shaft 12.

The previously described embodiments include various elements which could also be combined in instruments in a different manner. For example, the differently formed processing tools 24 may also be used respectively for other embodiments. Additionally, elements, such as the light sheath 50, can correspondingly be used in other embodiments, in particular also with the flexible endoscope shaft 4. The arrangement of two rinse channels 68 and 70, which is shown in FIG. 14, may also be used in the other embodiments described.

Further, in the above-described examples the hollow shaft 12 is always described as a rotationally driven component. Alternatively or additionally, the hollow shaft 12 can also perform a translational, oscillating movement in the direction of the longitudinal axis.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. An endoscope comprising a mobile hollow shaft (12) extending in a longitudinal direction (X) of the endoscope, a processing tool (24) arranged on a distal end of the endoscope and being mobile via the mobile hollow shaft (12), and an endoscope optic (14) arranged inside the mobile hollow shaft (12).

2. The endoscope according to claim 1, wherein the hollow shaft (12) is mobile in the longitudinal direction (X) and/or rotates around the longitudinal axis (X).

3. The endoscope according to claim 1, wherein the hollow shaft (12) has a circular cross-section.

4. The endoscope according to claim 1, wherein bearing elements (18, 20) are arranged between an inner wall of the hollow shaft (12) and an outer wall of the endoscope optic (14).

5. The endoscope according to claim 1, wherein a drive (10) is provided at a proximal end of the endoscope for moving the hollow shaft (12).

6. The endoscope according to claim 1, wherein the hollow shaft (12) has a flexible form.

7. The endoscope according to claim 1, wherein the endoscope optic (14) has a flexible form.

8. The endoscope according to claim 1, wherein a coupling is provided at a distal end of the hollow shaft (12) for receiving the processing tool (24).

9. The endoscope according to claim 8, further comprising a transmission (46) at the distal end of the hollow shaft (12) for moving the processing tool (24).

10. The endoscope according to claim 9, wherein the transmission (46) comprises an angle drive.

11. The endoscope according to claim 1, wherein at least one probe channel (36) is formed on the hollow shaft (12) extending in the longitudinal direction (X) of the endoscope.

12. The endoscope according to claim 1, wherein the processing tool (24) is formed directly at a distal end of the hollow shaft (12).

13. The endoscope according to claim 1, wherein the processing tool (24) is formed as at least one of a saw, a cutter, and a grinding tool.

14. The endoscope according to claim 1, wherein the hollow shaft (12) is removable from the endoscope.

15. The endoscope according to claim 1, wherein the endoscope optic (14) is formed as a lens system, a fiber optic, or a camera system.

16. The endoscope according to claim 1, wherein at least one recess (30) is formed on a distal end wall of the hollow shaft (12) located in a view field (28) of the endoscope optic (14) or can be moved into the view field (28) of the endoscope optic (14).

17. The endoscope according to claim 16, wherein the at least one recess (30) is formed in a circumferential wall of the hollow shaft (12) and the view field (28) of the endoscope optic (14) is at least partially aligned in a radial direction relative to the longitudinal axis (X) of the endoscope.

18. The endoscope according to claim 16, wherein an image is captured by the endoscope optic (14) via a camera, and the image capturing is synchronized with movements of the hollow shaft (12).

19. The endoscope according to claim 1, further comprising at least one rinse channel (16; 68; 70) formed between an outer wall of the endoscope optic (14) and an inner wall of the hollow shaft (12).

20. The endoscope according to claim 1, further comprising a light sheath (50) for illuminating an area surrounding the distal end of the endoscope.

21. The endoscope according to claim 20, wherein the light sheath (50) is arranged outside the hollow shaft (12).

22. The endoscope according to claim 20, wherein the light sheath (50) comprises a light guide.

23. The endoscope according to claim 20, wherein at least one lighting means is arranged in the light sheath (50).

24. The endoscope according to claim 20, wherein the light sheath (50) is movable together with the hollow shaft (12).

25. The endoscope according to claim 20, wherein the light sheath (50) is movable relative to the hollow shaft (12).

26. The endoscope according to claim 1, wherein the hollow shaft (12) is surrounded circumferentially by a protective sheath (42).
